(19) United States
(12) Patent Application Publication
(10) Pub. No.: US 2022/0000473 A1
(43) Pub. Date: Jan. 6, 2022

(54) METHOD AND DEVICE FOR VESICOURETHRAL ANASTOMOSIS

(71) Applicant: Mayo Foundation for Medical Education and Research, Rochester, MN (US)

(72) Inventors: Rachel Mann, Homer Glen, IL (US); Madeline Smerchansky, Arlington, VA (US); Nicholas Quan, Atlanta, GA (US); Bailey Klee, Atlanta, GA (US); Raymond W. Pak, Atlanta, GA (US); Jared M. Brown, Jefferson, FL (US)

(21) Appl. No.: 17/289,921

(22) PCT Filed: Dec. 3, 2019
(86) PCT No.: PCT/US19/64167

§ 371 Date: Apr. 29, 2021

Related U.S. Application Data
(60) Provisional application No. 62/774,498, filed on Dec. 3, 2018.

(51) Int. Cl. A61B 17/04; A61B 17/062; A61B 17/11

(52) U.S. Cl. CPC A61B 17/0469; A61B 2017/00278; A61B 17/11; A61B 17/062

(57) ABSTRACT

This document describes devices and methods for suturing an anastomosis. For example, this document describes transurethral probe devices that can be used to suture a vesicourethral anastomosis with one continuous stitch along a helical toroidal path.

### CROSS-REFERENCE TO RELATED APPLICATIONS

This application claims the benefit of U.S. Provisional Application Ser. No. 62/774,498, filed Dec. 3, 2018. The disclosure of the prior application is considered part of (and is incorporated by reference in) the disclosure of this application.

### BACKGROUND

### 1. Technical Field

This document relates to devices and methods for suturing an anastomosis. For example, this document relates to a trans-urethral probe device that can be used to complete a vesicourethral anastomosis with one continuous stitch along a helical toroidal path.

### 2. Background Information

One in nine men will develop prostate cancer at some point in their lifetime, making prostate cancer the second leading cause of cancer death among men in the United States. The most common treatment for prostate cancer is a prostatectomy, which is the complete surgical removal of the prostate. Every year in the United States, there are 90,000 prostatectomies performed, of which approximately 90 percent are done using tele-operated robotic surgery systems. Over $1.1 billion is spent on U.S. prostatectomy procedures annually.

During a prostatectomy, the entire prostate is cut out including the section of the urethra that runs through it. FIG. 1 shows the male anatomy prior to the prostatectomy. FIG. 2 shows the anatomy after removal of the prostate. In order to restore urinary function to the patient after removing the prostate, the surgeon must reconnect the urethra to the bladder using a procedure known as the vesicourethral anastomosis.

Currently, surgeons spend an average of 34 minutes hand sewing the bladder and urethra back together. During this part of the procedure, varying patient anatomies along with poor visibility and restricted maneuverability inside of the pelvic cavity can make the hand stitching anastomosis process difficult for surgeons to perform, resulting in a wide variety of surgical outcomes. If the junction is not properly sewn, severe complications, such as urethral stricture formation, tissue necrosis, bladder neck contractures, urinary incontinence, prolonged catheter usage, and life-threatening infections, can arise. One in three men who undergo a prostatectomy experience these completely preventable complications.

Postoperative complications associated with prostatectomy are an undue drain on the resources and time of patients and hospitals, costing $142M annually to treat. Patients experiencing complications require five or more catheters each day, and these catheters and their packaging take a huge toll on the environment, contributing up to 235,400 pounds of non-recyclable waste every day. In addition to the environmental toll, catheter use costs patients and providers an estimated $1.51B annually.

### SUMMARY

This document describes devices and methods for suturing an anastomosis. For example, this document describes trans-urethral probe devices that can be used to suture a vesicourethral anastomosis with one continuous stitch along a helical toroidal path.

In one aspect, this disclosure is directed to a medical suturing device that includes a handle including an actuator, an elongate shaft extending distally from the handle and defining a longitudinal axis, and an end effector attached to a distal end portion of the shaft. The end effector includes an arcuate needle, a needle driver defining a needle track in which the arcuate needle is movably received, and a needle drive mechanism coupled to the needle driver. In response to an actuation of the actuator: (i) the needle driver rotates about the longitudinal axis and (ii) the needle drive mechanism propels the arcuate needle relative to the needle track.

Such a medical suturing device may optionally include one or more of the following features. The end effector may also include an outer collar that is fixedly coupled to the distal end portion of the shaft and that the needle driver is rotatably housed within. The outer collar may define openings through which the arcuate needle moves in response to the actuation of the actuator. The arcuate needle and the needle track may be helices with arc lengths of less than 360 degrees. The needle driver may rotate about the longitudinal axis by at least 20 degrees in response to the actuation of the actuator. The arcuate needle may have an arc radius that is greater than a radius of the shaft. The radius of the shaft may be 4 mm or less. The needle track may define a first open end and a second open end. In response to the actuation of the actuator, a portion of the arcuate needle may: (i) leave the needle track via the first open end, (ii) travels along a path outside of the needle track, and (iii) re-enter the needle track via the second open end. In some embodiments, the path outside of the needle track is a helical toroidal path. The arcuate needle may include a plurality of unidirectional barbs.

In another aspect, this disclosure is directed to a method of suturing an anastomosis juncture between two anatomical structures. The method includes advancing a probe device into a lumen of a first anatomical structure of the two anatomical structures until a distal end portion of the probe device extends beyond an end of the first anatomical structure and into a lumen of a second anatomical structure of the two anatomical structures, and actuating an actuator of the probe device one or more times to drive an arcuate needle of the probe device to puncture each of the two anatomical structures multiple times to create a single continuous running stitch around a periphery of the juncture of the two anatomical structures.

Such a method may optionally include one or more of the optional features. Actuating the actuator of the probe device may drive the actuate needle along a helical toroidal path to create the single continuous running stitch. The first anatomical structure may be a urethra and the second anatomical structure may be a bladder. Actuating the actuator of the probe device may cause a tip portion of the arcuate needle to: (i) extend transversely away from the probe device, (ii) travel along a helical toroidal path, and (iii) return transversely toward the probe device.

In another aspect, this disclosure is directed to a medical suturing device that includes a handle including an actuator, an elongate shaft extending distally from the handle, and an end effector attached to a distal end portion of the shaft. The end effector includes a needle driver defining a needle track configured to movably receive a suturing needle, and a needle drive mechanism coupled to the needle driver and operable for driving a suturing needle within the needle track in response to actuation of the actuator. The needle track extend along a helical path.

Such a medical suturing device may optionally include one or more of the following features. The needle driver may rotate about a longitudinal axis of the shaft in response to the actuation of the actuator. The end effector may also include an outer collar that is fixedly coupled to the distal end portion of the shaft and that the needle driver is rotatably housed within. The outer collar may define openings through which the suturing needle passes in response to the actuation of the actuator. The medical suturing device may also include the suturing needle. The suturing needle may be helically shaped. The needle track may define a first open end and a second open end. In response to the actuation of the actuator, a portion of the suturing needle may: (i) leave the needle track via the first open end, (ii) travels along a path outside of the needle track, and (iii) re-enter the needle track via the second open end.

Particular embodiments of the subject matter described in this document can be implemented to realize one or more of the following advantages. With the use of the vesicourethral anastomosis probe devices described herein, surgeons can semi-automatically stitch the bladder and urethra back together with the simple pull of a trigger. Accordingly, surgeons will no longer need to manipulate suture needles manually, thus enhancing the efficiency of the procedure and eliminating complications that arise from poor visibility and restricted maneuverability. In addition, the anastomosis suturing devices described herein standardize the procedure across surgeons of varying experience levels, and eradicate both the time-consuming frustrations and variability associated with hand sewing. The precision and reliability with which the anastomosis suturing devices described herein operate greatly reduces patients' risk for complications. With the use of the vesicourethral anastomosis probe devices described herein, patients can expect better surgical outcomes, and therefore faster recovery times and higher quality of life post-surgery. Moreover, the helical needle and rotational mechanism of the anastomosis suturing probe devices described herein help to standardize the completion of the vesicourethral anastomosis, which is currently extremely variable in number of stitches placed and distance of their placement to each orifice. This standardization will help to reduce postoperative complications that are associated with a poorly sewn anastomosis. The anastomosis suturing probe devices described herein are the first suturing devices that can be used in associate with a minimally invasive prostatectomy performed using a surgical robotic system. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 shows a portion of male anatomy including the urethra, prostate, and bladder.

FIG. 2 shows the anatomy of FIG. 1 after removal of the prostate.

FIG. 3 shows an example use of an example transluminal anastomosis-suturing device in accordance with some embodiments provided herein.

FIGS. 4A-4C depict the progressive movement of an arcuate needle of an end effector of the transluminal anastomosis-suturing device of FIG. 3 as the needle moves along a helical toroid path to create a sutured anastomosis between a urethra and bladder.

FIG. 5 is an isometric view of a needle driver component of the end effector of the transluminal anastomosis-suturing device of FIG. 3. Arrows depict the rotational movement of the needle driver about the longitudinal axis of the instrument shaft and the helical movement of the arcuate needle.

FIG. 6 is an enlarged front view of the face of the needle driver of FIG. 5 showing a needle track defined by the needle driver.

FIG. 7 illustrates the 360-degree helical toroidal path followed by the helical needle of the transluminal anastomosis-suturing device of FIG. 3 as suturing is performed using the device.

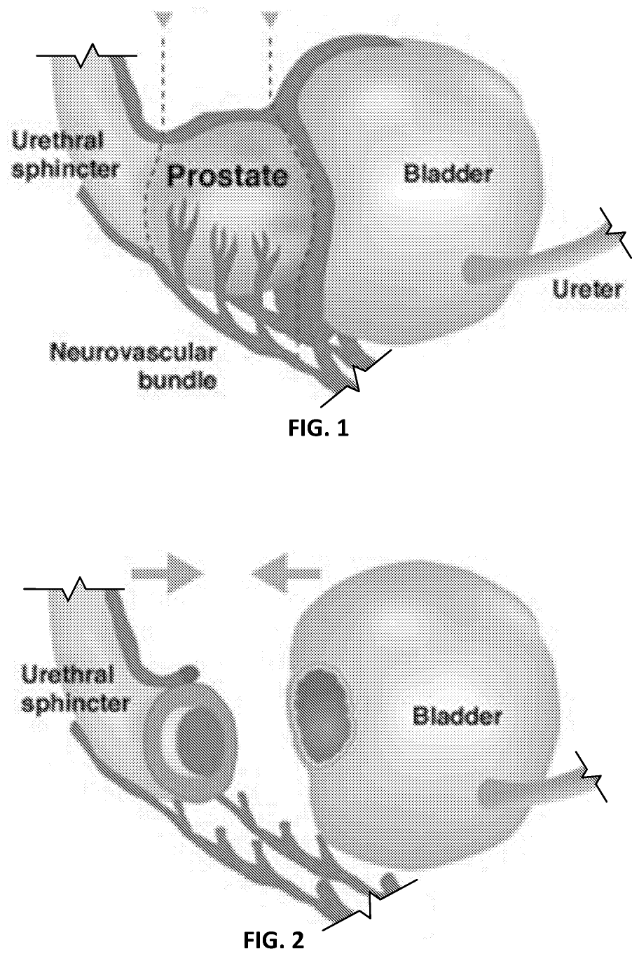

Like reference numbers represent corresponding parts throughout.

### DETAILED DESCRIPTION

This document describes devices and methods for suturing an anastomosis. For example, this document describes trans-urethral probe devices that can be used to suture a vesicourethral anastomosis with one continuous stitch along a helical toroidal path.

While the devices and methods are described in the context of a vesicourethral anastomosis, it should be understood that the devices and methods described herein may also be used to suture anastomoses between other anatomical structures such as, but not limited to, a colorectal anastomosis, an esophageal anastomosis, vein anastomosis, anastomoses in the gastro-intestinal tract, and others. The devices described herein are scalable to an appropriate size to suit the dimensions of the anatomical structures to be anastomosed.

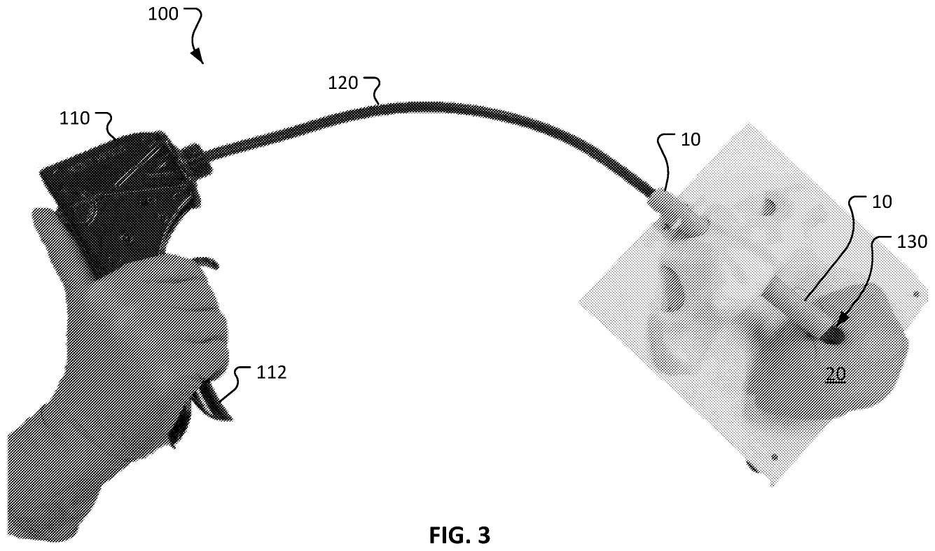

As shown in FIG. 3, an example transluminal anastomosis-suturing device 100 in accordance with this disclosure can be used to create a sutured anastomosis juncture between two anatomical structures. In the depicted example, a sutured vesicourethral anastomosis is being created between a urethra 10 and a bladder 20 after a prostatectomy procedure.

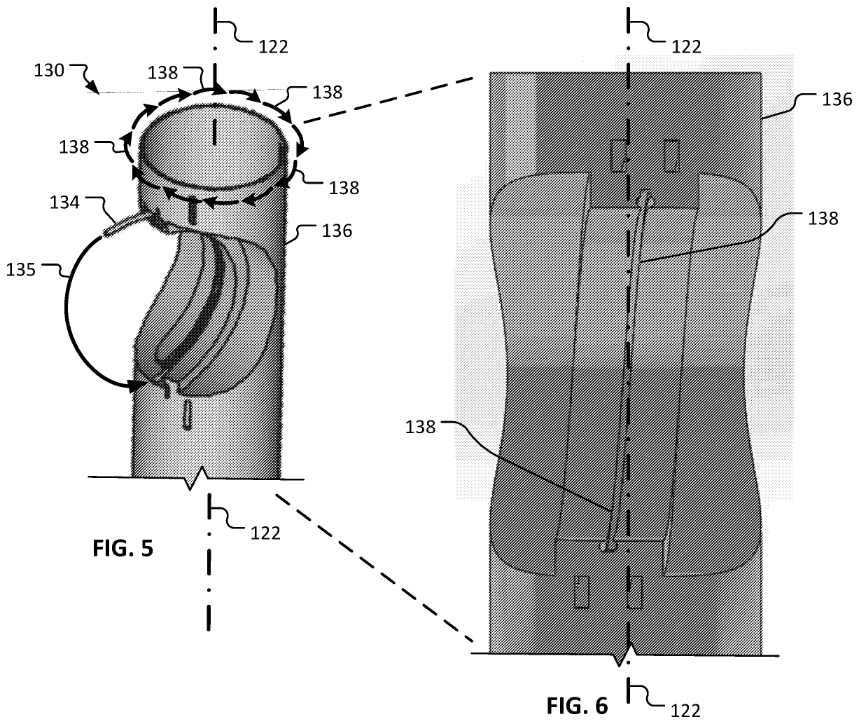

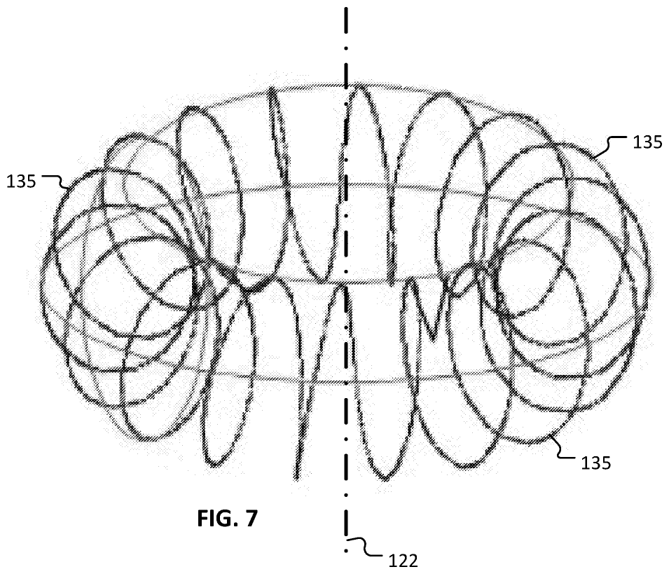

The transluminal anastomosis-suturing device 100 includes a handle 110 with an actuator 122, an elongate shaft 120 extending distally from the handle 110 and defining a longitudinal axis 122 (FIGS. 5 and 6), and an end effector 130 attached to a distal end portion of the shaft 120.

In brief, and as explained further below, the transluminal anastomosis-suturing device 100 is a probe device that can install sutures. The device 100 can be inserted, for example, into the penis via the urethra 10 and advanced toward the bladder 20. Once the end effector 130 of the transluminal anastomosis-suturing device 100 is spanning the anastomotic junction between the severed end of the urethra 10 and the ostomy of the bladder 20 (e.g., by about 5 mm of each), a clinician actuates the actuator 112 (e.g., pulls a trigger), driving an arcuate helical needle attached to a suture looping through both bladder 20 and urethral tissues 10, placing one helical stitch. This action is then repeated until the device 100 has placed a single continuous stitch around the entire periphery of the anastomotic junction (e.g., 360 degrees) between the urethra 10 and the bladder 20. In some embodiments, without limitation, the transluminal anastomosis-suturing device 100 is used in conjunction with a minimally invasive tele-operated robotic surgery system.

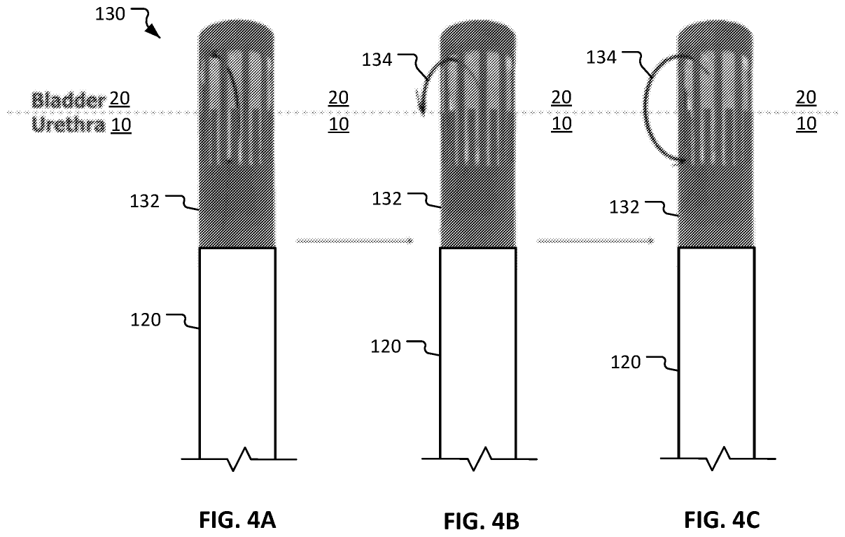

Now also referring to FIGS. 4A-4C, the end effector 130 includes an outer collar 132 that is fixedly coupled to the distal end portion of the device shaft 120. In response to an actuation of the actuator 112, an arcuate suture needle 134 of the end effector 130 is driven along a helical path as illustrated by the progressive sequence of FIGS. 4A through 4C.

The outer collar 132 of transluminal anastomosis-suturing device 100 described herein, which comes in contact with the inside of the bladder and urethral tissues, is stationary during suturing to prevent tissue damage. The outer collar 132 defines openings (e.g., slits) through which the arcuate needle 134 and attached suture move in response to the actuation of the actuator 112. The openings of the outer collar 132 are positioned and shaped to align with the path of the needle 134 to ensure that the outer collar 132 does not become attached to the anatomical structures by the suture attached to the needle 134.

Now also referring to FIG. 5, here the end effector 130 is shown without the outer collar 132 so that an inner needle driver 136 that is housed within the outer collar 132 is visible. As the actuator 112 is actuated, each time: (i) the needle driver 136 incrementally pivots, indexes, or rotates about the longitudinal axis 122 as depicted by individual arrows 138 and (ii) a needle drive mechanism coupled to the needle driver 136 propels the arcuate needle 134 along a single helical cycle of a helical toroidal path 135 (see also FIG. 7). Each incremental rotational motion of the needle driver 136 about axis 122 is accompanied by a single complete helical cycle of the actuate needle 134. In other words, when the needle driver 136 has been caused to rotate a full 360 degrees about the longitudinal axis 122 (e.g., in response to multiple actuations of the actuator 112), the actuate needle 134 has completed multiple helical cycles (as depicted in FIG. 7), which result in creating a single continuous running stitch around a periphery of the juncture of the two anatomical structures.

In the depicted embodiment, a total of twelve manual actuations of the actuator 112 are required to incrementally rotate the needle driver 136 a full 360 degrees about the longitudinal axis 122. Accordingly, in this embodiment the needle driver 136 incrementally rotates 30 degrees in response to each single actuation of the actuator 112. As the actuator 112 is actuated additional times, the arcuate needle 134 punctures through the wall of each of the two anatomical structures each time to create a single continuous running stitch around a 360 degree periphery of the juncture of the two anatomical structures. The single continuous running stitch follows along a helical toroidal path (as depicted in FIG. 7).

In some embodiments, rotational increments of the needle driver 136 other than 30 degrees of rotation in response to each actuation of the actuator 112 can be used. For example, in some embodiments the incremental rotation of the needle driver 136 in response to each actuation of the actuator 112 can be, for example, 15 degrees, 20 degrees, 40 degrees, 45 degrees, 60 degrees or anywhere in between those values.

By using a unique design of a helical needle 134 in accordance with this disclosure, it is ensured that the ending point of one stitch is the start of the next. In the depicted example embodiment, the helical needle 134 is offset at the correct angle to ensure that exactly 12 sutures are placed around the 360-degree junction of the urethra and bladder. The needle 134 is shaped helically, with an arcuate diameter of 10 mm, to ensure an optimal needle puncture placement of 5mm from the meeting of the urethra and bladder tissues. This amount of suture throw and distance of placement is optimal to prevent tissue tearing and necrosis. In combination with the helical needle 134, the incremental rotational movement of the needle driver 136 was created to allow for continuous stitching along the helical toroidal path 135 (see FIG. 7).

In some embodiments, the needle 134 is driven by a needle drive mechanism that includes rotating friction wheels (e.g., similar to those found on a Bowden extruder). In some embodiments, the friction wheels can be rigidly attached to concentric gears that are driven by a central shaft and gear. In some such embodiments, the needle 134 is pushed and/or pulled by these friction wheels along a needle track 138 that is defined by the needle driver 136 (see FIG. 6). In the depicted embodiment, the needle track 138 extends along a helical path that corresponds to the helical shape of the needle 134.

In some embodiments, the outer diameter of the shaft 120 and the outer collar 132 of the transluminal anastomosis-suturing device 100 described herein are about 8 mm. This aligns with the average size of the male urethra in order for the device to work for the majority of the population. However, in some embodiments the shaft 120 and the outer collar 132 can be between 7 mm and 9 mm, or between 6 mm and 10 mm.

It should be understood that the shaft 120 and the outer collar 132 are scalable to an appropriate scale to suit the size of the anatomical structures to be anastomosed. For example, when the transluminal anastomosis-suturing device 100 is scaled for colorectal anastomosis procedures, the outer diameter of the shaft 120 and the outer collar 132 may be between 1.0 inch (2.5 cm) and 3.0 inches (7.6 cm), or between 2.0 inches (5.1 cm) and 3.0 inches (7.6 cm), or between 1.5 inches (3.8 cm) and 2.5 inches (6.4 cm), without limitation. In another example, when the transluminal anastomosis-suturing device 100 is scaled for esophageal anastomosis procedures, the outer diameter of the shaft 120 and the outer collar 132 may be between 0.5 inches (1.3 cm) and 1.0 inch (2.5 cm), or between 0.5 inches (1.3 cm) and 0.75 inches (1.9 cm), or between 0.75 inches (1.9 cm) and 1.0 inch (2.5 cm), or between 0.75 inches (1.9 cm) and 1.25 inches (3.2 cm), without limitation.

In the depicted embodiment, the arcuate needle track 138 of transluminal anastomosis-suturing device 100 extends along a helical arc with a diameter that is slightly larger than 10 mm to guide the arcuate helical needle 134 having a 10 mm arc diameter. As the shaft 120 and the outer collar 132 of transluminal anastomosis-suturing device 100 described herein is about 8 mm in diameter (i.e., smaller than the arcuate diameter of the needle 134) this means that the pathway of the needle 134 must overlap within the device, necessitating the need for a track 138 that rotates as the needle does 134. This is one reason why the needle driver 136 with its needle track 138 rotates around the longitudinal axis 122 to complete the entire helical-toroidal pathway of the needle 134, as required to create the single continuous running stitch around a periphery of the juncture of the two anatomical structures. The resulting single continuous running stitch around the periphery of the juncture of the two anatomical structures follows a helical toroidal path as depicted in FIG. 7. In the depicted embodiment, the helical needle 134 is used in conjunction with transluminal anastomosis-suturing device 100 described herein to precisely and reliably place 12 stitches (which is actually a single continuous running stitch) around the periphery of the juncture of the two anatomical structures.

The helical needle 134 has a specific arcuate diameter in order to ensure that each stitch is placed a set distance from the ends of the two tissues. In the case of the vesicourethral anastomosis, the optimal distance to place the stitch from the meeting point of the two tissues is 5 mm, so the helical needle 134 has an arcuate diameter of 10 mm. According to medical textbooks, the optimal number of stitches is to place around the vesicourethral anastomotic site is 12, and the diameter of the shaft 120 of the transluminal anastomosis-suturing device 100 is 8 mm, giving the helical needle 134 a helical pitch of 2.09 mm. This shape of the helical needle 134 allows the end of one stitch to be the start of the next, creating a running stitch. In some embodiments, the helical needle 134 has a different helical pitch.

In some embodiments, the helical needle 134 has several small unidirectional barbs along its outer side that can be used by the needle drive mechanism within the needle driver 136 to move the needle 134. In some embodiments, the suture attached to the needle 134 will also be barbed, in order to prevent slippage between the tissues as it creates a continuous stitch (in some embodiments sutures that are currently used in this procedure are also barbed).

In some embodiments, the helical needle 134 is inserted into the needle driver 136 within the body (in situ), after the transurethral insertion of the transluminal anastomosis-suturing device 100 itself. That is, in some cases the transluminal anastomosis-suturing device 100 is inserted into the urethra 10, and once in sight in the junction between the bladder 20 and the urethra 10, the helical needle 134 is then inserted through the abdominal port (for robotic procedures) or surgical site (for open procedures). The helical needle 134 is then loaded by the surgeon into the needle driver 136, which is accomplished by rotating it into the needle track 138 of the needle driver 136. However, in some embodiments the helical needle 134 is pre-loaded in the needle driver 136 and then the transluminal anastomosis-suturing device 100 is inserted into the urethra 10.

### Additional Optional Features and/or Embodiments

Some embodiments of the transluminal anastomosis-suturing devices described herein can include an additional feature to hold the bladder and urethra stump abutted together in preparation for the anastomosis suturing process.

Some embodiments of the transluminal anastomosis-suturing device described herein complete one single helical cycle stitch in response to one actuation of the actuator. Alternately, some embodiments require two or more actuations to complete a single helical cycle stitch. In another alternative embodiment, the entire 360 degree helical-toroidal running stitch can be completed in response to a single actuation of the actuator. In some embodiments, the transluminal anastomosis-suturing device is electrically powered.

In some embodiments, the helical needle has an arc of about 270 degrees. In some embodiments, the arcuate needle has an arc between about 180 degrees and 270 degrees, or between 200 degrees and 270 degrees, or between 220 degrees and 270 degrees, or between 240 degrees and 270 degrees, or between 220 degrees and 250 degrees, or between 200 degrees and 230 degrees, without limitation.

In some embodiments, the needle drive mechanism includes a gear train and other components such as, but not limited to, one or more rotating friction wheels, a central driving shaft (e.g., flexible drive shaft), and a driving mechanism. For example, the needle can be driven by a plurality of rotating friction wheels that are coupled to driven gears or axles.

In some embodiments, the needle drive mechanism can include a spring that “catches” the barbed needle. In this iteration, there can be a channel containing a spring inside the needle driver. The channel has an opening along the needle track through which the needle's barbs protrude. The spring is attached to a wire that is attached to the actuator. When the actuator is actuated, the spring compresses and propels the needle through the needle track.

In some embodiments, the suture connected to the helical needle is a barbed suture, to help ensure a tight connection is created and maintained.

The transluminal anastomosis-suturing devices described herein are able to satisfy user needs through unique features and benefits. The transluminal anastomosis-suturing devices described herein are precise—placing a set number of stitches a set distance apart, standardizing the quality of the anastomosis reconnection. In some embodiments, the transluminal anastomosis-suturing device described herein evenly places 12 stitches around the periphery that are at a distance of 5 mm from each orifice.

The transluminal anastomosis-suturing devices described herein are unique in that the sutures are placed from the inside out, where the current methods require the surgeons to place sutures from the outside in.

### Example Method of Use (Vesicourethral Anastomosis)

1. After removal of the prostate, the surgeon performs posterior reconnection to approximate the bladder and urethra.

2. The transluminal anastomosis-suturing device described herein is inserted into the urethra in a manner similar to a catheter or probe. The surgeon advances the transluminal anastomosis-suturing device from the urethral opening to the gap between the bladder and urethra.

3. The helical needle is inserted into the abdomen via abdominal port.

4. The helical needle is loaded into the transluminal anastomosis-suturing device by the surgeon using the robotic arms.

5. The bladder and urethra stump are approximated over the end effector of the transluminal anastomosis-suturing device. The junction between the bladder and urethra is approximately centered on the outer collar 132 (as illustrated in FIGS. 4A-4C).

6. The surgeon actuates the actuator located at the handle of the transluminal anastomosis-suturing device, external to the body (as illustrated in FIG. 3). The actuation sends a helical needle in a 360-degree helical loop (a single helical cycle). The helical needle punctures through bladder and urethral tissue (each tissue is puncture twice) as illustrated in FIGS. 4A-4C, placing one helical stitch.

7. The surgeon repeats actuations as many times as needed (e.g., 12 times) to create a single continuous running stitch around a periphery of the juncture of the two anatomical structures (e.g., placing twelve stitches around the 360 degree bladder-urethral junction).

8. The surgeon cuts the suture to detach the helical needle from the suture, and ties the suture ends together, securing the suture.

9. The helical needle is withdrawn from the abdominal port.

10. The transluminal anastomosis-suturing device is retracted from the urethra and disposed of.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A medical suturing device comprising:
a handle including an actuator;
an elongate shaft extending distally from the handle and defining a longitudinal axis; and
an end effector attached to a distal end portion of the shaft and comprising:
an arcuate needle;
a needle driver defining a needle track in which the arcuate needle is movably received; and
a needle drive mechanism coupled to the needle driver,
wherein, in response to an actuation of the actuator: (i) the needle driver rotates about the longitudinal axis and (ii) the needle drive mechanism propels the arcuate needle relative to the needle track.

2. The medical suturing device of claim 1, wherein the end effector further comprises an outer collar that is fixedly coupled to the distal end portion of the shaft and that the needle driver is rotatably housed within.

3. The medical suturing device of claim 2, wherein the outer collar defines openings through which the arcuate needle moves in response to the actuation of the actuator.

4. The medical suturing device of claim 1, wherein the arcuate needle and the needle track are helices with arc lengths of less than 360 degrees.

5. The medical suturing device of claim 1, wherein the needle driver rotates about the longitudinal axis by at least 20 degrees in response to the actuation of the actuator.

6. The medical suturing device of claim 1, wherein the arcuate needle has an arc radius that is greater than a radius of the shaft.

7. The medical suturing device of claim 6, wherein the radius of the shaft is 4 mm or less.

8. The medical suturing device of claim 1, wherein the needle track defines a first open end and a second open end, and
wherein, in response to the actuation of the actuator, a portion of the arcuate needle: (i) leaves the needle track via the first open end, (ii) travels along a path outside of the needle track, and (iii) re-enters the needle track via the second open end.

9. The medical suturing device of claim 8, wherein the path outside of the needle track is a helical toroidal path.

10. The medical suturing device of claim 1, wherein the arcuate needle includes a plurality of unidirectional barbs.

11. A method of suturing an anastomosis juncture between two anatomical structures, the method comprising:
advancing a probe device into a lumen of a first anatomical structure of the two anatomical structures until a distal end portion of the probe device extends beyond an end of the first anatomical structure and into a lumen of a second anatomical structure of the two anatomical structures; and
actuating an actuator of the probe device one or more times to drive an arcuate needle of the probe device to puncture each of the two anatomical structures multiple times to create a single continuous running stitch around a periphery of the juncture of the two anatomical structures.

12. The method of claim 11, wherein actuating the actuator of the probe device rotates a friction wheel that drives the actuate needle along a helical toroidal path to create the single continuous running stitch.

13. The method of claim 11, wherein the first anatomical structure is a urethra and the second anatomical structure is a bladder.

14. The method of claim 11, wherein actuating the actuator of the probe device causes a tip portion of the arcuate needle to: (i) extend transversely away from the probe device, (ii) travel along a helical toroidal path, and (iii) return transversely toward the probe device.

15. A medical suturing device comprising:
a handle including an actuator;
an elongate shaft extending distally from the handle; and
an end effector attached to a distal end portion of the shaft and comprising:
a needle driver defining a needle track configured to movably receive a suturing needle; and
a needle drive mechanism coupled to the needle driver and operable for driving a suturing needle within the needle track in response to actuation of the actuator,
wherein the needle track extends along a helical path.

16. The medical suturing device of claim 15, the needle driver rotates about a longitudinal axis of the shaft in response to the actuation of the actuator.

17. The medical suturing device of claim 15, wherein the end effector further comprises an outer collar that is fixedly coupled to the distal end portion of the shaft and that the needle driver is rotatably housed within.

18. The medical suturing device of claim 17, wherein the outer collar defines openings through which the suturing needle passes in response to the actuation of the actuator.

19. The medical suturing device of claim 15, further comprising the suturing needle, and wherein the suturing needle is helically shaped.

20. The medical suturing device of claim 19, wherein the needle track defines a first open end and a second open end, and
wherein, in response to the actuation of the actuator, a portion of the suturing needle: (i) leaves the needle track via the first open end, (ii) travels along a path outside of the needle track, and (iii) re-enters the needle track via the second open end.
